(19) United States
(12) Patent Application Publication
(10) Pub. No.: US 2010/0140111 A1
(43) Pub. Date: Jun. 10, 2010

(54) METHOD AND ARRANGEMENT FOR ELECTRICALLY CONTACTING AN OBJECT SURROUNDED BY A MEMBRANE, USING AN ELECTRODE

(75) Inventors: Jan Gimsa, Rostock (DE); Ulrike Gimsa, Rostock (DE); Stefan Fiedler, Berlin (DE); Torsten Müller, Berlin (DE); Wolfgang Scheel, Berlin (DE)

Correspondence Address: FOLEY AND LARDNER LLP;SUITE 500, 3000 K STREET NW, WASHINGTON, DC, 20007, US

(73) Assignees: FRAUNHOFER-GESELLSCHAFT ZUR FÖRDERUNG DER ANGEWANDTEN FORSCHUNG E.V., München (DE); FORSCHUNGSINSTITUT FÜR DIE BIOLOGIE LANDWIRTSCHAFTLICHER NUTZTIERE, Dummerstorf (DE)

(21) Appl. No.: 12/451,059

(22) PCT Filed: Mar. 31, 2008
(86) PCT No.: PCT/DE2008/000568

§ 371 Date: Feb. 4, 2010

(30) Foreign Application Priority Data
Apr. 25, 2007 (DE) 10 2007 019 842.8

(51) Int. Cl. G01N 27/26; C12N 13/00; C25B 11/02

(52) U.S. Cl. 205/777.5; 435/173.4; 204/290.01

(57) ABSTRACT

Method and arrangement for making electrical contact with a membrane-enveloped object using an electrode

The invention relates, inter alia, to a method for making electrical contact with a membrane-enveloped object (30) using an electrode (10, 100).

According to the invention, it is provided that at least one electrode (100) comprising a conductive carrier (110) is used for making contact, on which carrier a multiplicity of nanoneedles (120) are arranged and on which carrier adjacent nanoneedles are at a distance from one another which is smaller than the size of the object, and that the object is brought into contact with the nanoneedles.

Method and arrangement for making electrical contact with a membrane-enveloped object using an electrode

At the present time, possibilities for electrical stimulation and/or tapping of electrical signals from biological cells or tissues are the subject of intensive research. The aim is to achieve as low-impedance coupling as possible between the cell or tissue and a conductive electrode.

While traditional patch clamp measuring techniques detect measurement signals only via individual membrane fragments (so-called patches) and channels situated therein and thus permit statements about intact cells in the physiological state only to a limited extent, further-developed whole cell clamp techniques (known as: whole cell voltage clamping, whole cell patch clamp) are disadvantageous insofar as they are always accompanied by a cell penetration (through a capillary or directly through an electrode) and hence breaching of the cell membrane. The low-impedance connection to the capillary or its counterpart requires special precautions owing to which automation or measurements over relatively long periods of time is/are often at least made more difficult. As is known, the exclusively capacitive detection of electrophysiological signals from individual cells, cell assemblages (tissue sections) or tissues is made more difficult by high leakage current proportions and inadequate signal coupling-in.

A generally poor electrical and mechanical coupling between electrode and cell or tissue arises in the case of purely external tapping e.g. in multielectrode arrays (MEAs) as a result of the generally relatively large distance of on average greater than 40 nm between electrode and cell and the influence of the electrical double layers in the aqueous phase both on the electrode surface and on the cell membrane. In the case of the current flow required for the electrical signal transmission, direct-current or low-frequency components lead to disadvantageous electrochemical processes at the surfaces and in the aqueous phase; such electrochemical processes lead to distortions of applied or tapped-off electrical signals.

Proceeding from the prior art outlined above, the invention is based on the object of specifying a method for making electrical contact with a membrane-enveloped object, such as a biological cell, for example, in the case of which a lowest possible coupling impedance between the membrane-enveloped object and the electrode is achieved.

This object is achieved according to the invention by means of a method comprising the features in accordance with claim 1. Advantageous configurations of the method are specified in dependent claims.

Accordingly, it is provided according to the invention that at least one electrode comprising a conductive carrier is used for making contact, on which carrier a multiplicity of nanoneedles are arranged and on which carrier adjacent nanoneedles are at a distance from one another which is smaller than the size of the membrane-enveloped object, and that the membrane-enveloped object is brought into contact with the nanoneedles. The membrane-enveloped object can be, for example, a biological (human, animal or vegetable) cell, a liposome, a lipid film (e.g. black lipid membrane) or a structure having a multilamellar construction.

The shaping of the nanoneedles is as desired, moreover; the nanoneedles can have any desired cross section (round, angular, oval, etc.) and any desired ratio between length and width: thus, the nanoneedles can be longer than they are wide or alternatively wider than they are long. By way of example, they can be column- or lobe-shaped and form nanorods or nanowires. The form of the “needle tip” or of the needle end face can also be configured in highly varied fashion: by way of example, the needle end face can have a burr or taper to a point.

One essential advantage of the method according to the invention is that a very intimate contact between electrode and object and thus a very low contact resistance or contact impedance are achieved on account of the nanoneedles arranged at the surface of the electrode. Whereas cells settle on smooth planar surfaces generally at a distance of at least 40 nm from the surface, a significantly smaller distance is achieved in the case of the electrode used according to the invention, as a result of which the electrical contact resistance or contact impedance can be reduced and the tapping or read-out of electrical measurement signals can be effected with higher accuracy than in previous contact-making methods.

A further essential advantage of the method according to the invention can be seen in the fact that the contact-making is non-invasive despite the presence of needles; this can be attributed inter alia to the fact that the needles are configured as nanoneedles and, moreover, are at a distance from one another which is smaller than the size of the object. This arrangement additionally has the effect that the object sinks between the nanoneedles without the membrane of the membrane-enveloped object being damaged or penetrated in the process.

A third advantage of the method according to the invention can be seen in the fact that, owing to the use of the “nanoneedle-decorated” electrode described, the mapping of the electrical cell activity or the stimulation is possible with very few errors in both spatially and temporally resolved fashion. Furthermore, impedance characteristics of adherently growing cells can be detected very precisely under physiological conditions.

Preferably, the needle tips of the “nanolawn” formed by the nanoneedles constitute focal contact points at which the distance between membrane and needle surface is less than 10 nm, to be precise without the membrane being penetrated. As a result of the smallness of the membrane contact areas with respect to the nanoneedle tip, special molecular structures are formed, in particular in cells in the membrane or in direct proximity to the membrane, and they support the intimate contact between the membrane and the needle surface. The contact reliability is improved further on account of the high attractive interaction forces as a result of the small distance (e.g. van der Waals force). This can lead to the formation of anisotropic membrane regions.

Preferably, an electrode is used in the case of which the nanoneedles on the carrier are distributed irregularly, in particular stochastically, at least in sections. This is because if the nanoneedles on the carrier are distributed irregularly or stochastically and if they thus form at least in part areas of needles or needle groups adjacent to one another at different distances, then cell-physiologically beneficial effects are additionally induced: this is because, in contrast to strictly symmetrical nanoneedle arrays, an overstimulation that can lead to a stress situation (e.g. phagocytosis induction by carbon nanotubes) and hence to unphysiological conditions is generally avoided in the case of irregularly or stochastically arranged nanoneedles.

Particularly preferably, an electrode is used in the case of which the nanoneedles on the carrier are distributed irregularly, in particular stochastically, in at least one section and are distributed regularly in at least one other section. A change between regions with regular needle arrangement and those with irregular needle arrangement ensures good nestling of the object against the carrier and additionally simplifies automatic, for example computer-aided, recognition of the electrode regions and thus automatic, in particular optical, characterization of the cells.

The electrode can also be formed solely by a substrate on which cells can grow.

The nanoneedles can be metallic (mono- or polycrystalline), for example. In this case, the nanoneedles and the carrier can consist of the same or of different materials; by way of example, the carrier and/or the nanoneedles can consist of a noble metal, preferably gold or platinum, a base metal, preferably titanium, a conductive, nonconductive or poorly conductive polymer or a semiconductor material or comprise such a material.

Moreover, it is regarded as advantageous if a nanoneedle-carrying surface needles of a delimited region are electrically connected at the surface and form one electrode, wherein adjacent needles either can be assigned to another electrode or are not electrically contact-connected toward the outside. In the case of the last-mentioned embodiment, therefore, by way of example, at least one needle section with which electrical contact can be made and at least one needle section with which electrical contact cannot be made are combined with one another.

If the nanoneedles consist of a conductive material, then it is regarded as advantageous if the radii of curvature of the needle end faces or needle tips are so small that they can operate as field emitters; suitable needle tip diameters are of the magnitude of between 10-25 nm and 1-2 μm.

Particularly good nestling of the object against the carrier and thus a particularly small distance between carrier and membrane-enveloped object can be achieved if an electrode is used in the case of which the nanoneedles are nonconductive or at least more poorly conductive than the conductive carrier. In the case of such a configuration of the electrode, a very low contact resistance occurs even though the nanoneedles themselves are nonconductive or are only poorly conductive; in this case, the nanoneedles nevertheless contribute to the reduction of the contact resistance because they promote the nestling of the cell against the conductive carrier and thus reduce the distance between carrier and cell.

Preferably, an electrode is used in the case of which the distance between adjacent nanoneedles is on average (averaged over the number of nanoneedles) less than 10 μm and/or on average less than one hundred times the nanoneedle diameter. The size indication relates to biological cells of average size having a diameter of 3-50 μm. In the case of larger cells, the distance can also be correspondingly enlarged. The nanoneedles preferably have a diameter of between 10 nm and 1200 nm, preferably between 50 and 800 nm. The length of the nanoneedles preferably lies between 100 nm and 20 micrometers, particularly preferably between 300 nm and 10 micrometers.

The nanoneedles can also have a coating in order to further improve the contact with the object or to achieve a local assignment. The coating of the nanoneedles with molecules (non-specifically e.g. polylysine, specifically with receptors and/or ligands) can additionally improve the mechanical and electrical coupling of the membrane to the needles. In this case, the molecules can reach into the membrane and/or through it.

The contact-making method described is preferably used in the context of a method for carrying out electrical measurements on a membrane-enveloped object and/or for the stimulation of a membrane-enveloped object, wherein contact is made with the object in the manner described, and then electrical measurement signals of the object are measured by means of the electrode and/or a stimulation of the object is carried out by applying an electrical voltage or by electric current.

The methods described can be used for example for signal tapping—and/or for electrical stimulation, i.e. bidirectionally:
on cells of the nervous system or electrically excitable cells, such as e.g. muscle cells, muscle parts, tissue, wherein particular importance is accorded to nerve cells and the myocardium,
in biohybrid systems,
in interfaces between microelectronic components and living cells and tissues,
for the purpose of signal tapping on electrically active cells or electrically stimulatable or excitable cells or multicell systems, e.g. muscle cells and/or cells of the nervous system such as neurons, neuronal networks, microglial cells, oligodendrocytes and/or astrocytes,
for the purpose of measurements on and/or with artificial cell-like structures which are enveloped e.g. by a phospholipid membrane which should not be breached, for instance on liposomes, vesicles or more complexly shaped compartments enveloped by a single- or multilayered molecular layer (e.g. block copolymer membranes), or lipid-protein layers (e.g. black lipid membranes),
for applying electrical signals (different frequencies, in particular pulsed and RF signals), to living cells and tissues, and
in human-machine interfaces.

The methods described can also be employed for example:
for the facilitated electrofusion of living cells under “milder” conditions, in particular of cells which otherwise form hybrids only with difficulty or with an inadequate yield, or of mixed cell types (e.g. adherent feeder layer and suspension cells), one or both of which grow(s) adherently,
for the facilitated electroporation of cells for the improved yield of transfected cells,
for the low-loss (e.g. capacitive) coupling of cell body and electrode surface with reduced leakage current proportion, without breaching or penetrating the cell membrane in the process,
for the improved integral impedance measurement on cells, in 96-well plates, such as are offered commercially for example by Applied Biophysics, USA,
for avoiding the influencing of the measurement signal by electrode processes (minimizing electrochemical surface reactions on the electrode and on the coupled biological membrane or surface),
for prosthetics: control of prostheses or muscles with the aid of neural signals,
for implants: improved biocompatibility of electrode areas and surfaces of sensory components,
for cell-based biosensors, e.g. in cell sensor chips,
for electrically induced cell-cell, cell-vesicle, vesicle-vesicle fusion (electrofusion) and
for fundamental cell-biological and/or medical research; e.g. in so-called neurosensor chips.

The invention additionally relates to an electrode suitable for making electrical contact with a membrane-enveloped object, in particular a biological cell (human, animal or vegetable cell).

According to the invention, it is provided that the electrode has a conductive carrier, on which a multiplicity of nanoneedles are arranged and on which adjacent nanoneedles are at a distance from one another which is smaller than the size of the membrane-enveloped object, in particular smaller than a biological cell.

With regard to the advantages of the electrode according to the invention and with regard to the advantages of advantageous configurations of the electrode according to the invention, reference should be made to the explanations above in connection with the method according to the invention.

The invention additionally relates to an arrangement comprising a plurality of electrodes, for example to a multielectrode array, wherein a plurality of electrodes of the type described are arranged two-dimensionally or three-dimensionally, for example in array-like fashion.

It holds true, for example, that contact can be made with one cell by a plurality of electrodes or with a plurality of cells by one electrode or with exactly one cell by one electrode. This furthermore facilitates an individual assignment of the signals to a cell.

An apparatus for carrying out electrical measurements on a membrane-enveloped object and/or for electrically stimulating a membrane-enveloped object is also regarded as an invention provided that it has one or more electrode(s) of the type described.

The invention is explained in more detail below on the basis of exemplary embodiments; in this case, by way of example:

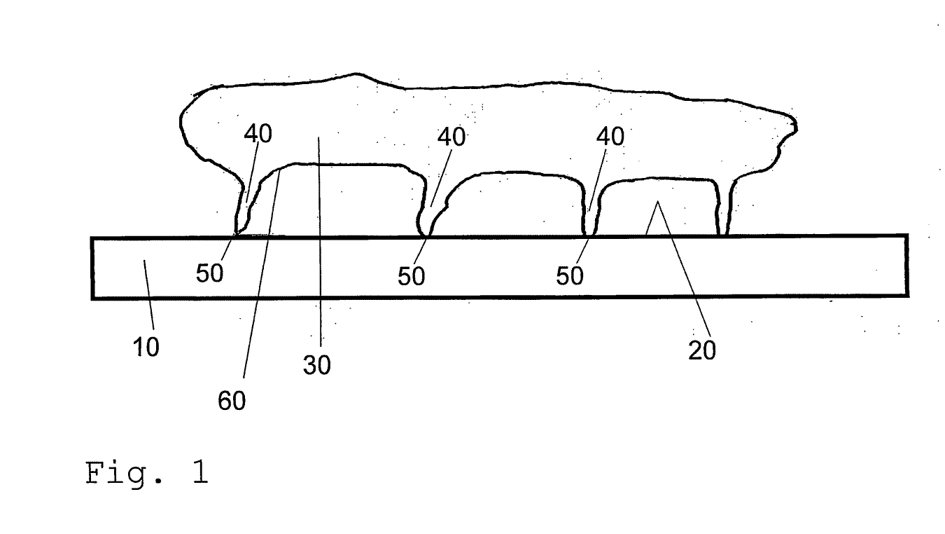

FIG. 1 shows, for general elucidation, an electrode without nanoneedles, with a biological cell situated on it,

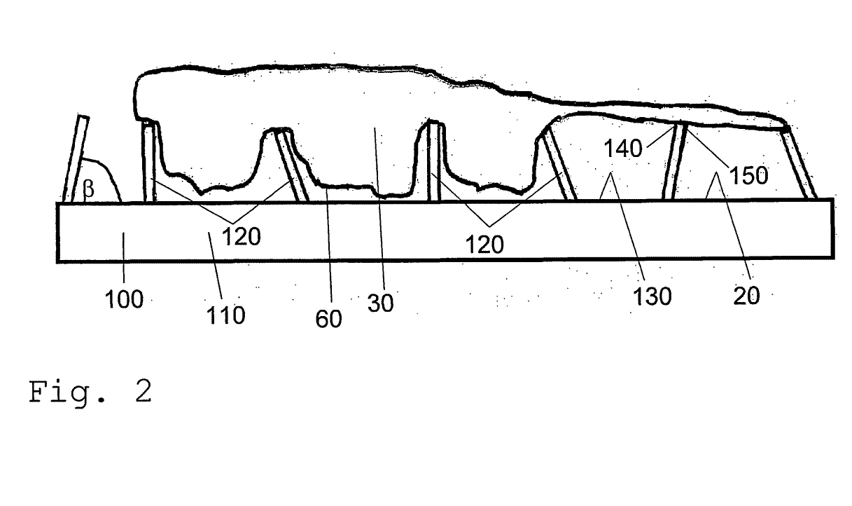

FIG. 2 shows a first exemplary embodiment of an electrode according to the invention with nanoneedles,

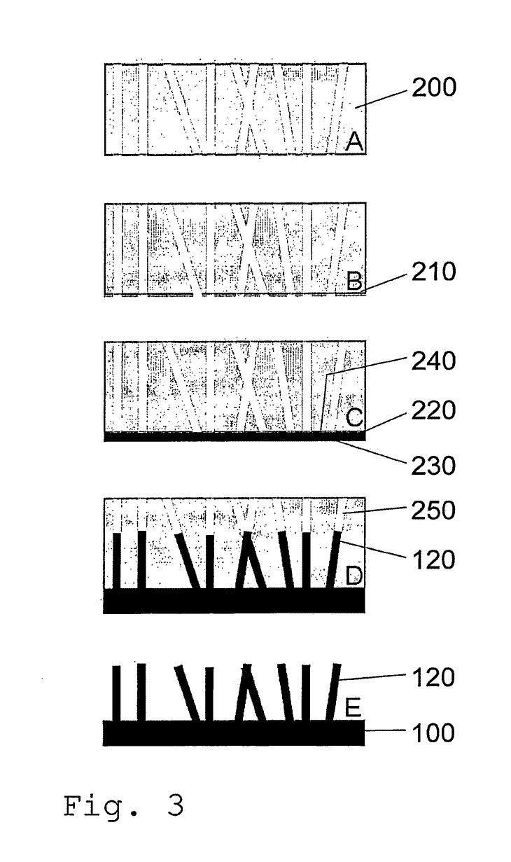

FIG. 3 shows an exemplary embodiment of the production of the electrode in accordance with FIG. 2,

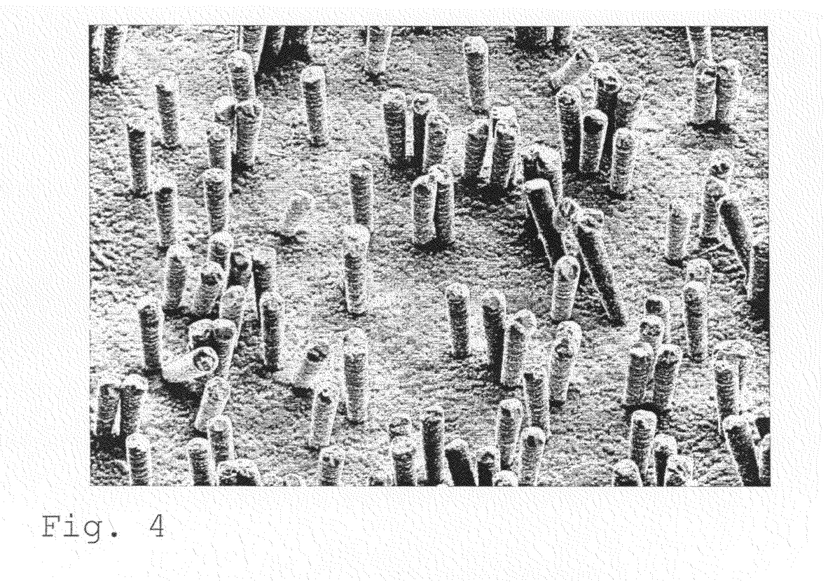

FIG. 4 shows by way of example a micrograph, recorded by an electron microscope, of an electrode according to the invention with carrier and nanoneedles,

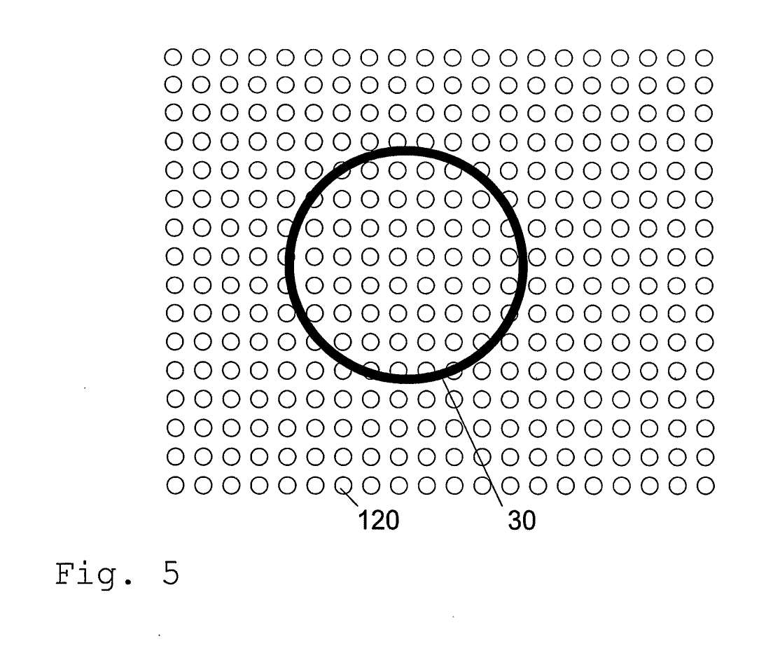

FIG. 5 schematically shows an exemplary embodiment of an electrode according to the invention with a regular or symmetrical nanoneedle distribution,

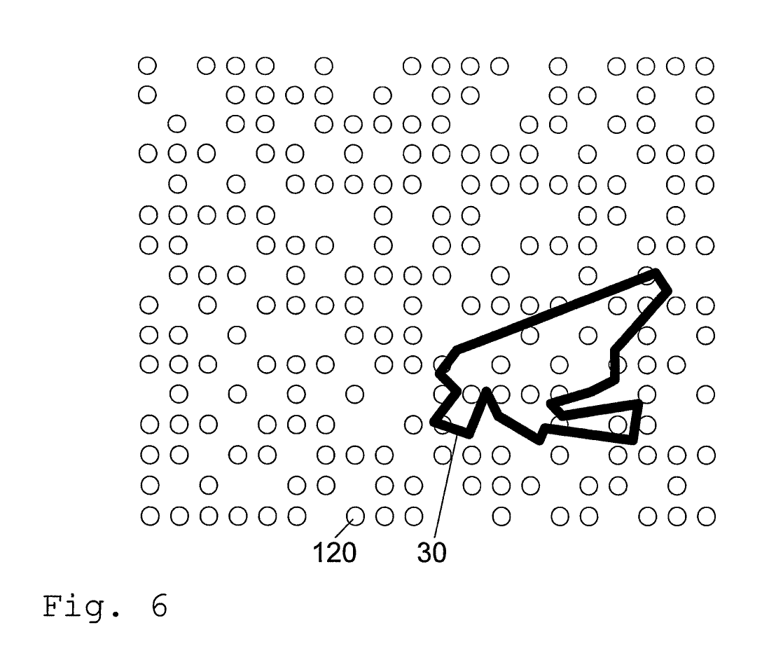

FIG. 6 schematically shows an exemplary embodiment of an electrode according to the invention with an irregular or stochastic nanoneedle distribution,

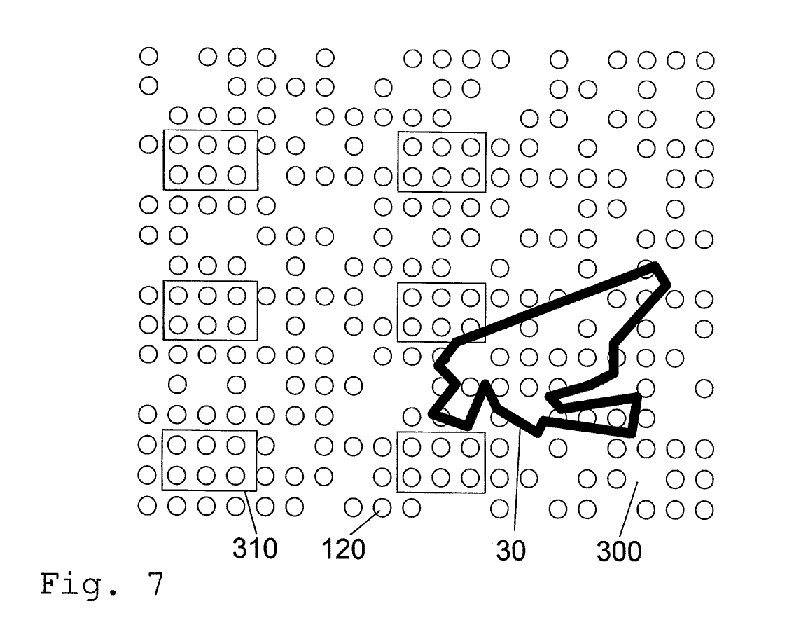

FIG. 7 schematically shows an exemplary embodiment of an electrode according to the invention with nanoneedle sections with an irregular or stochastic nanoneedle distribution and nanoneedle sections with a regular or symmetrical nanoneedle distribution, and

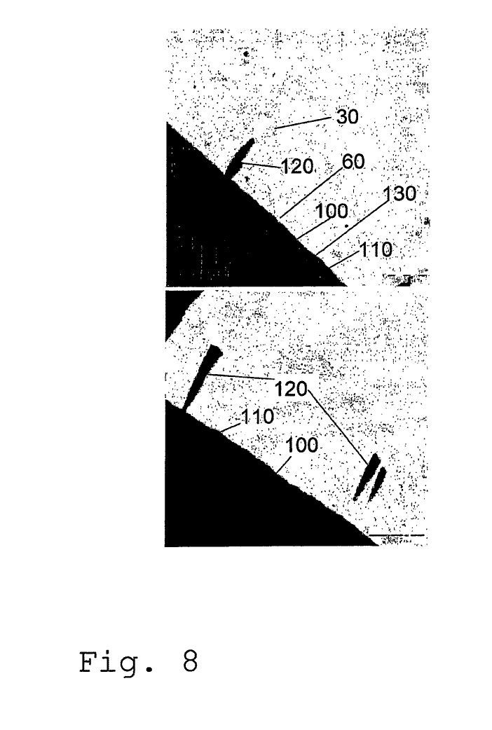

FIG. 8 shows a micrograph, recorded by transmission electron microscopy, of a cell arranged on an exemplary embodiment of an electrode according to the invention.

In FIGS. 1 to 8, the same reference symbols are always used for identical or comparable components.

FIG. 1 shows, for general elucidation, an electrode 10 with a smooth electrode surface 20 without nanoneedles. A biological (human, animal or vegetable) cell 30 with which contact is made by means of the electrode 10 forms focal contact points 50 with the electrode 10 by means of membrane protuberances 40. The distance between the membrane 60 of the cell 30 and the smooth electrode surface 20 is on average (averaged over the membrane area facing the electrode 10) typically greater than 40 nm.

FIG. 2 shows an exemplary embodiment of an electrode 100 according to the invention. The electrode 100 has a carrier 110 and nanoneedles 120 oriented partly perpendicularly (angle β=90° and partly angularly (angle β<90° with respect to the surface 130 of the carrier 110. The nanoneedles 120 form on the carrier a “nano-lawn”, which has been produced for example using nanoimprint techniques, semiconductor technology and/or by electrolytic deposition.

The distance between directly adjacent nanoneedles is preferably smaller than the size of the cell 30. Focal contact points 140 between the cell 30 and the electrode 100 are formed at the needle tips 150. The nanoneedles 120 result in a nestling of the cell against the surface 130 of the carrier 110 and thus on average a smaller distance between the membrane 60 of the cell 30 and the electrode surface 20 than in the case of the electrode 10 without nanoneedles in accordance with FIG. 1. Typically, the distance between the membrane 60 of the cell 30 and the surface 130 of the carrier 110 in the case of an electrode like that in accordance with FIG. 2 is on average less than 5 nm.

The angular orientation of the nanoneedles 120 is preferably set in such a way that the nanoneedles have in sections or “in populations” similar angles β with respect to the surface 130 of the carrier 110. Preferably, the angular deviation of the angles in one and the same section of the carrier 110 is less than 20 degrees, preferably less than 10 degrees.

FIG. 8 shows a micrograph, recorded by transmission electron microscopy, of a cell 30 arranged on an electrode 100. The intimate contact between the surface 130 of the carrier 110 and the membrane 60 of the cell 30 can be discerned.

FIG. 3 illustrates by way of example, on the basis of five illustrations A to E, how the electrode 100 in accordance with FIG. 2 can be produced. The topmost illustration A reveals a nanoporous polymer film 200, which is subjected to sputtering on one side on the underside and coated with a thin electrically conductive layer 210 (cf. illustration B). An electrodeposition of a layer serving as working electrode 220 is subsequently carried out (illustration C). During the electrodeposition, deposition occurs not only on the underside 230 of the layer 210, but also on the top side 240, on which the nanoporous polymer film 200 bears. In this case, the growth takes place through the pores 250 of the nanoporous polymer film 200, whereby the nanoneedles 120 are formed (illustration D).

After the conclusion of the needle growth, the nanoporous polymer film 200 is removed, for example by a solvent or by etching, whereby the electrode 100 with the nanoneedles 120 is completed (illustration E).

The nanoporous polymer film 200 can be for example a nanoporous polymer template, also called “nuclear track membrane” or “track etched membranes”. The nanoporous polymer film 200 can be produced by irradiating a polymer film with high-energy particles and expanding the disturbances present in latent fashion after the irradiation in the polymer film using suitable etchants to form the continuous pores 250.

Depending on the etching time, the etching media and further parameters, it is possible to produce very defined pore widths in the range of from 10 nm to more than 5 μm, even up to 10 μm. The density of the pores per unit area can be configured in different ways by means of the conditions of the primary particle bombardment.

In order to achieve different needle angles β, the polymer film 200 is for example irradiated sequentially multiply at different angles and only then etched in one step.

FIG. 4 shows by way of example a micrograph, recorded by an electron microscope, of an electrode with carrier and with nanoneedles.

FIG. 5 schematically illustrates an exemplary embodiment with a regular or symmetrical nanoneedle distribution. It can be discerned that the symmetrical distribution of the nanoneedles induces a symmetrical shaping of the cell 30, which usually does not correspond to the physiological situation in vivo.

Therefore, an irregular or stochastic distribution of the nanoneedles is better than a regular or symmetrical nanoneedle distribution, such an irregular or stochastic distribution being illustrated as a further exemplary embodiment in FIG. 6. It can be discerned that the cell 30 adapts to the nanoneedle distribution, whereby even better nestling against the carrier 110 is achieved and the distance between the cell 30 and the carrier 110 is reduced even further.

In order to simplify automatic locating on the carrier 110 for automated cell recognition, it is regarded as advantageous if one or more nanoneedle sections with an irregular or stochastic distribution of the nanoneedles and one or more nanoneedle sections with a regular or symmetrical nanoneedle distribution are present or combined with one another; such an exemplary embodiment is shown in FIG. 7. The cells will nestle well against the carrier 110 in the nanoneedle sections 300 with the irregular or stochastic distribution of the nanoneedles 120, and the nanoneedle sections 310 with the regular or symmetrical distribution of the nanoneedles 120 simplify automatic image processing.

### REFERENCE SYMBOLS

10 Electrode
20 Electrode surface
30 Biological cell
40 Membrane protuberances
50 Contact points
60 Membrane
100 Electrode
110 Carrier
120 Nanoneedles
130 Surface of the carrier
140 Focal contact points
150 Needle tips
200 Polymer film
210 Electrically conductive layer
220 Conductive layer
230 Underside
240 Top side
250 Pores
300 Nanoneedle section with irregular or stochastic distribution of the nanoneedles
310 Nanoneedle section with regular or symmetrical di1stribution of the nanoneedles
β Angle between nanoneedle and surface of the carrier

1. A method for making electrical contact with a membrane-enveloped object using an electrode,
wherein at least one electrode comprising a conductive carrier is used for making contact, on which carrier a multiplicity of nanoneedles are arranged and on which carrier adjacent nanoneedles are at a distance from one another which is smaller than the size of the object,
wherein the object is brought into contact with the nanoneedles, and
wherein the nanoneedles on the carrier are distributed irregularly, in particular stochastically, in at least one section and are distributed regularly in at least one other section.

2. The method as claimed in claim 1, characterized in that the object with which contact is made is a biological cell, a biological tissue, a liposome, a lipid film or a structure having a multilamellar construction.

3. The method as claimed in claim 1, characterized in that the contact-making is non-invasive.

4. The method as claimed in claim 1, characterized in that the nanoneedles are lobe-shaped.

5. The method as claimed in claim 1, characterized in that an electrode is used in the case of which the nanoneedles are nonconductive or more poorly conductive than the carrier.

6. The method as claimed in claim 1, characterized in that an electrode is used in the case of which the distance between adjacent nanoneedles is on average less than one hundred times the nanoneedle diameter.

7. The method as claimed in claim 1, characterized in that an electrode is used in the case of which the nanoneedles have a diameter of between 10 nm and 1200 nm.

8. The method as claimed in claim 1, characterized in that an electrode is used in the case of which the nanoneedles have a length of between 100 nm and 20 micrometers.

9. The method as claimed in claim 1, characterized in that the carrier and/or the nanoneedles consist of a noble metal, preferably gold or platinum, a base metal, preferably titanium, a conductive, nonconductive or poorly conductive polymer or a semiconductor material or comprise such a material.

10. The method as claimed in claim 1, characterized in that a sensing tip with a plurality of nanoneedle arrays is used as the electrode.

11. The method as claimed in claim 1, characterized in that the object is coupled to at least two electrodes provided with nanoneedles.

12. The method as claimed in claim 1, characterized in that the cells are grown on the electrode in the context of making contact.

13. The method as claimed in claim 1, characterized in that the electrode of a neurosensor chip is used.

14. The method as claimed in claim 1, characterized in that the nanoneedles on the carrier form a nanolawn that has been produced using nanoimprint techniques, semiconductor technology and/or by electrolytic deposition.

15. The method as claimed in claim 1 wherein electrical measurements are carried out on said membrane-enveloped object and/or a stimulation of the membrane-enveloped object is made, wherein electrical measurement signals of the object are measured by means of the electrode and/or a stimulation of the object is carried out by applying an electrical voltage or by electric current.

16. An electrode suitable for making electrical contact with a membrane-enveloped object,
wherein the electrode has a conductive carrier,
on which a multiplicity of nanoneedles are arranged and
on which adjacent nanoneedles are at a distance from one another which is smaller than the size of the object, and
wherein the nanoneedles on the carrier are distributed irregularly, in particular stochastically, in at least one section and are distributed regularly in at least one other section.

17. The electrode as claimed in claim 16, characterized in that the nanoneedles are lobe-shaped.

18. (canceled)

19. The electrode as claimed in claim 16, characterized in that the nanoneedles are nonconductive or more poorly conductive than the carrier.

20. The electrode as claimed in claim 16, characterized in that the distance between adjacent nanoneedles is on average less than one hundred times the nanoneedle diameter.

21-29. (canceled)

30. A method for making electrical contact with a membrane-enveloped object using an electrode,
wherein at least one electrode comprising a conductive carrier is used for making contact, on which carrier a multiplicity of nanoneedles are arranged and on which carrier adjacent nanoneedles are at a distance from one another which is smaller than the size of the object,
wherein the object is brought into contact with the nanoneedles, and
wherein the nanoneedles are lobe-shaped.
